SEQUENCE LISTING (5 sequences)

SEQ ID NO: 1 - protein, 396 aa, Homo sapiens
MVAGTRCLLALLLPQVLLGGAAGLVPELGRRKFAAASSGRPSSQPSDEVLSEFELRLLSMFGLKQRPTPSRDAVVPPYMLDLYRRHSGQPGSPAPDHRLERAASRANTVRSFHHEESLEELPETSGKTTRRFFFNLSSIPTEEFITSAELQVFREQMQDALGNNSSFHHRINIYEIIKPATANSKFPVTRLLDTRLVNQNASRWESFDVTPAVMRWTAQGHANHGFVVEVAHLEEKQGVSKRHVRISRSLHQDEHSWSQIRPLLVTFGHDGKGHPLHKREKRQAKHKQRKRLKSSCKRHPLYVDFSDVGWNDWIVAPPGYHAFYCHGECPFPLADHLNSTNHAIVQTLVNSVNSKIPKACCVPTELSAISMLYLDENEKVVLKNYQDMVVEGCGCR

SEQ ID NO: 2 - protein, 23 aa, Artificial
  Synthetic construct
KIPKASSVPTELSAISTLYLDDD

SEQ ID NO: 3 - protein, 28 aa, Artificial
  Synthetic construct
CCCCDDDSKIPKASSVPTELSAISTLYL

SEQ ID NO: 4 - protein, 28 aa, Artificial
  Synthetic construct
CCCCGGGSKIPKASSVPTELSAISTLYL

SEQ ID NO: 5 - protein, 431 aa, Artificial
  Synthetic construct
MHVRSLRAAAPHSFVALWAPLFLLRSALADFSLDNEVHSSFIHRRLRSQERREMQREILSILGLPHRPRPHLQGKHNSAPMFMLDLYNAMAVEEGGGPGGQGFSYPYKAVFSTQGPPLASLQDSHFLTDADMVMSFVNLVEHDKEFFHPRYHHREFRFDLSKIPEGEAVTAAEFRIYKDYIRERFDNETFRISVYQVLQEHLGRESDLFLLDSRTLWASEEGWLVFDITATSNHWVVNPRHNLGLQLSVETLDGQSINPKLAGLIGRHGPQNKQPFMVAFFKATEVHFRSIRSTGSKQRSQNRSKTPKNQEALRMANVAENSSSDQRQACKKHELYVSFRDLGWQDWIIAPEGYAAYYCEGECAFPLNSYMNATNHAIVQTLVHFINPETVPKPCCAPTQLNAISVLYFDDSSNVILKKYRNNVVRACGCH

(19) United States
(12) Patent Application Publication
(10) Pub. No.: US 2022/0133957 A1
(43) Pub. Date: May 5, 2022

(54) Sclerostin Inhibitors That Promote Bone Morphogenetic Protein Expression

(71) Applicants: Emory University, Atlanta, GA (US); The United States Government as Represented by the Department of Veterans Affairs, Washington, DC (US)

(72) Inventors: Scott D. Boden, Atlanta, GA (US); Steven M. Presciutti, Decatur, GA (US); Sreedhara Sangadala, Marietta, GA (US)

(21) Appl. No.: 17/428,556

(22) PCT Filed: Feb. 4, 2020
(86) PCT No.: PCT/US2020/016529

§ 371 Date: Aug. 4, 2021

Related U.S. Application Data
(60) Provisional application No. 62/800,743, filed on Feb. 4, 2019.

(51) Int. Cl. A61L 27/54; A61K 31/506; A61K 31/19; A61L 27/24; A61L 27/52; A61L 27/22

(52) U.S. Cl. CPC A61L 27/54; A61K 31/506; A61K 31/19; A61L 27/24; A61L 27/52; A61L 2300/204; A61L 2300/414; A61L 2300/252; A61L 2430/02; A61L 2430/06; A61L 2300/21; A61L 27/227

(57) ABSTRACT

This disclosure relates to sclerostin inhibitors for use in ossification, and methods related thereto. In certain embodiments, the disclosure relates to placing sclerostin inhibitors in graft compositions for forming bone. In certain embodiments, the disclosure relates to methods of forming bone comprising implanting a graft composition disclosed herein optionally comprising a growth factor such as BMP or recombinant vector expressing the same in a subject such as at a desired site of bone or cartilage growth.

### CROSS-REFERENCE TO RELATED APPLICATIONS

This application claims the benefit of U.S. Provisional Application No. 62/800,743 filed Feb. 4, 2019. The entirety of this application is hereby incorporated by reference for all purposes.

### STATEMENT REGARDING FEDERALLY SPONSORED RESEARCH OR DEVELOPMENT

This invention was made with government support under BX003845 awarded by the U.S. Department of Veterans Affairs. The government has certain rights in the invention.

### INCORPORATION-BY-REFERENCE OF MATERIAL SUBMITTED AS A TEXT FILE VIA THE OFFICE ELECTRONIC FILING SYSTEM (EFS-WEB)

The Sequence Listing associated with this application is provided in text format in lieu of a paper copy and is hereby incorporated by reference into the specification. The name of the text file containing the Sequence Listing is 19012PCT_ST25.txt. The text file is 9 KB, was created on Feb. 3, 2020 and is being submitted electronically via EFS-Web.

### BACKGROUND

Bone grafting is a surgical procedure to repair missing or fractured bone. Bone grafting is typically performed for spinal fusions, after cancerous bone removal, and in certain operations, e.g., plastic surgery. In autologous grafting, the iliac crest is often used as a donor site; however, complications can arise including pain, nerve damage, hematoma and wound complications, avulsion of the anterior superior iliac spine (ASIS), herniation of the abdominal cavity contents, and cosmetic deformity. Thus, it is desirable to develop materials and methods of forming bone that do not require harvesting bone from remote sites of the patient.

Synthetic bone grafts typically include a matrix that holds minerals and other salts. Natural bone has an intracellular matrix mainly composed of type I collagen, and some synthetic bone grafts include a collagen matrix. Synthetic bone grafts typically contain bone growths factors, such as bone morphogenetic proteins (BMPs), because of their ability to induce ossification in the matrix material. Recombinant human BMP-2 has been approved by the FDA in synthetic bone grafts such as INFUSE™. INFUSE™ is approved for open tibial shaft fractures, lumbar interbody fusion, and sinus and alveolar ridge augmentations. However, the high cost and need for high concentrations of BMP-2 for treatment creates a barrier for routine clinical use. Thus, there is a need to identify additional compositions that may substitute or complement the use of BMPs in treating bone-related conditions.

Wnt signaling regulates bone mass. See Krishnan et al. J Clin Invest, 2006, 116(5): 1202-1209. Wnt molecules bind to the Frizzled receptors and to the LRP5/6 co-receptors, thereby controlling the stability of cytoplasmic β-catenin. The importance of Wnt signaling in bone formation is illustrated by the low bone mass osteoporosis-pseudoglioma syndrome or high bone mass phenotype caused either by missense loss or gain of function mutations in LRP5, respectively. The regulation of the Wnt pathway is regulated physiologically by the Wnt receptor antagonist sclerostin, which binds to the LRP5/6 receptor and inactivates Wnt signaling. Therefore, sclerostin leads to the direct inhibition of osteoblastogenesis. McClung reports that sclerostin antibodies are potential therapeutics for treating osteoporosis. Ther Adv Musculoskel Dis, 2017, 9(10):263-270.

Boden et al. report compositions for ossification. See U.S. Pat. Nos. 9,808,464, 9,511,071, 9,295,754 and U.S. Patent Applications 2016/0361470, 2015/0374694, 2015/0148292, 2014/0248372, 2013/0344165, and 2013/0137634.

References cited herein are not an admission of prior art.

### SUMMARY

This disclosure relates to sclerostin inhibitors for use in ossification, and methods related thereto. In certain embodiments, the disclosure relates to placing sclerostin inhibitors in graft compositions for forming bone. In certain embodiments, the disclosure relates to methods of forming bone comprising implanting a graft composition disclosed herein optionally comprising a growth factor such as BMP or recombinant vector expressing the same in a subject such as at a desired site of bone or cartilage growth.

In certain embodiments, the disclosure relates to methods of forming bone comprising implanting a graft composition comprising a growth factor, such as BMP, in a subject at a site of desired bone growth or enhancement in combination with a sclerostin inhibitor disclosed herein in the bone graft composition and/or by administering a pharmaceutical composition comprising a sclerostin inhibitor disclosed herein to the subject. The sclerostin inhibitor may be used by itself without exogenous growth factor.

In certain embodiments, the disclosure relates to methods of forming bone comprising a) implanting a graft composition optionally comprising a sclerostin inhibitor disclosed herein and optionally comprising a growth factor in a subject at a site of desired bone growth and b) administering a pharmaceutical composition comprising a sclerostin inhibitor disclosed herein to the subject.

In certain embodiments, the sclerostin inhibitor is the compound 6-((2-(pyrimidin-2-ylamino)ethyl)amino)nicotinonitrile (C07), ester, derivative, or salt thereof. In certain embodiments, the derivative comprises one or more substituents.

In certain embodiments, the sclerostin inhibitor is valproic acid (VA1), ester, derivative, or salt thereof. In certain embodiments, the derivative comprises one or more substituents.

In certain embodiments, the sclerostin inhibitor is fluticasone (F), ester, derivative, or salt thereof, such as fluticasone propionate and fluticasone furoate. In certain embodiments, the derivative comprises one or more substituents.

In some embodiments, the disclosure relates to graft compositions comprising a sclerostin inhibitor disclosed herein, such as the compounds 6-((2-(pyrimidin-2-ylamino)ethyl)amino)nicotinonitrile (C07), valproic acid (VA1), fluticasone (F), or derivatives, and a graft matrix. Typically, the matrix comprises a collagen sponge and/or a compression resistant type I collagen and calcium phosphates. In other embodiments, the matrix is a hydrogel. In certain embodiments, the sclerostin inhibitor disclosed herein, such as the compounds 6-((2-(pyrimidin-2-ylamino)ethyl)amino)nicotinonitrile (C07), valproic acid (VA1), fluticasone (F), or derivatives, is covalently linked to a graft matrix.

Within certain embodiments, it is contemplated that the sclerostin inhibitors disclosed herein may be linked, e.g., covalently bound to the matrix, carrier, or scaffold such that a bone morphogenetic protein would be resistant to the degrading effects of the compound in order to reduce or eliminate the use of a bone morphogenetic protein in the graft composition to induce bone growth.

In certain embodiments, the bone graft compositions further comprise a bone morphogenetic protein and/or another growth factor. Typically, the bone morphogenetic protein is BMP-2 or BMP-7. In certain embodiments, the graft composition comprises calcium phosphates and/or bone granules, hydroxyapatite and/or beta-tricalcium phosphate, alpha-tricalcium phosphate, polysaccharides or combinations thereof. Crushed bone granules, typically obtained from the subject, are optionally added to the graft composition. In certain embodiments the graft further comprises cells capable of osteoblastic differentiation, such as mesenchymal stem cells and pre-osteoblastic cells. In certain embodiments, the graft further comprises a recombinant vector configured to express a growth factor or BMP.

In some embodiments, this disclosure relates to kits comprising a graft composition, a sclerostin inhibitor disclosed herein, such as the compounds 6-((2-(pyrimidin-2-ylamino)ethyl)amino)nicotinonitrile (C07), valproic acid (VA1), fluticasone (F), or derivatives and a graft matrix. In certain embodiments, the kits further comprise a bone morphogenetic protein and/or another growth factor or a recombinant vector that encodes a growth factor or BMP in operable combination with a promotor. In certain embodiments, the kits further comprise a transfer device, such as a syringe, nozzle, or pipette. In certain embodiments, the kit further comprises cells capable of osteoblastic differentiation, such as mesenchymal stem cells and pre-osteoblastic cells.

Compositions comprising sclerostin inhibitors disclosed herein may be dripped into the graft matrix, carrier, or scaffold optionally in combination with other osteogenic agents such as a mesenchymal stem cell, a bone morphogenetic protein, other bone growth factors and/or a statin.

In some embodiments, the disclosure relates to methods of generating BMP-mediated osteoblasts comprising administering an effective amount of a sclerostin inhibitor disclosed herein and cells capable of osteoblastic differentiation, such as mesenchymal stem cells and pre-osteoblastic cells.

In some embodiments, the disclosure relates to methods of forming bone comprising implanting a graft composition comprising a sclerostin inhibitor disclosed herein, such as the compounds 6-((2-(pyrimidin-2-ylamino)ethyl)amino)nicotinonitrile (C07), valproic acid (VA1), fluticasone (F), or derivatives, thereof in a subject under conditions such that bone forms in the graft. Typically, the subject has a void in the bony structure wherein the graft composition is implanted in the void. In certain embodiments, the void is in a bone selected from an extremity, maxilla, mandible, pelvis, spine and/or cranium. In certain embodiments, the void is a result of surgical removal of bone. In certain embodiments, the void is between bone and an implanted medical device. In another embodiment, the method further comprises the step of securing movement of bone structure with a fixation system, and removing the system after bone forms in the implanted graft.

In some embodiments, the disclosure relates to methods of performing spinal fusion comprising implanting a bone graft composition. The bone graft composition comprises a sclerostin inhibitor disclosed herein, such as the compounds 6-((2-(pyrimidin-2-ylamino)ethyl)amino)nicotinonitrile (C07), valproic acid (VA1), fluticasone (F), or derivatives configured to grow bone between two vertebrae of a subject. In certain embodiments, the composition further comprises a bone morphogenetic protein and/or another growth factor. In a typical embodiment, the subject is diagnosed with degenerative disc disease or has symptoms of back pain.

In some embodiments, the disclosure relates to methods of inserting a prosthetic device or anchor comprising, exposing the bone; implanting a graft composition comprising sclerostin inhibitors disclosed herein, such as the compounds 6-((2-(pyrimidin-2-ylamino)ethyl)amino)nicotinonitrile (C07), valproic acid (VA1), fluticasone (F), or derivatives in contact with the bone. In certain embodiments, one implants the prosthetic device or anchor in the graft composition. In certain embodiments, the composition further comprises a bone morphogenetic protein and/or another growth factor.

In some embodiments, the disclosure relates to pharmaceutical compositions comprising sclerostin inhibitors disclosed herein, such as the compounds 6-((2-(pyrimidin-2-ylamino)ethyl)amino)nicotinonitrile (C07), valproic acid (VA1), fluticasone (F), or derivatives, or pharmaceutically acceptable salts thereof. In certain embodiments, the compositions further comprise a bone morphogenetic protein and/or another growth factor. In certain embodiments, the pharmaceutical composition is formulated to release over a 12 hour, 1 day, 3 day, 5 day, 7 day, two week, or one month period.

In certain embodiments, the disclosure relates to methods of preventing or treating a bone fracture comprising administering a pharmaceutical composition, wherein the pharmaceutical composition comprises sclerostin inhibitors disclosed herein, such as the compounds 6-((2-(pyrimidin-2-ylamino)ethyl)amino)nicotinonitrile (C07), valproic acid (VA1), fluticasone (F), or derivatives or pharmaceutically acceptable salts thereof, to a subject at risk for, exhibiting symptoms of, or diagnosed with a bone fracture. In certain embodiments, the composition further comprises a bone morphogenetic protein and/or another growth factor. In certain embodiments, the administration is localized. In certain embodiments administration is achieved through oral delivery, intravenous delivery, parenteral delivery, intradermal delivery, percutaneous delivery, or subcutaneous delivery. In some embodiments, the method further comprises the step of exposing the bone fracture to pulsed electromagnetic fields. In further embodiments, the subject is diagnosed with a long bone shaft fracture such as the tibia or femur corrected with intramedullary nail fixation.

In some embodiments, the disclosure relates to methods of preventing or treating a bone degenerative disease comprising administering an effective amount of a pharmaceutical composition comprising sclerostin inhibitors disclosed herein, such as the compounds 6-((2-(pyrimidin-2-ylamino)ethyl)amino)nicotinonitrile (C07), valproic acid (VA1), fluticasone (F), or derivatives or pharmaceutically acceptable salts thereof, to a subject at risk for, exhibiting symptoms of, or diagnosed with a disease. In certain embodiments, the composition further comprises a bone morphogenetic protein and/or another growth factor. In certain embodiments, the administration is systemic or administration is achieved through oral delivery, intravenous delivery, parenteral delivery, intradermal delivery, percutaneous delivery, or subcutaneous delivery. In some embodiments, the disease is osteoporosis, osteitis deformans (Paget disease), bone metastasis, multiple myeloma, primary hyperparathyroidism, or osteogenesis imperfecta.

In some embodiments, the disclosure relates to methods for decreasing the time required to form new bone in the presence of a bone morphogenetic protein comprising co-administering at least one sclerostin inhibitor disclosed herein, such as the compounds 6-((2-(pyrimidin-2-ylamino)ethyl)amino)nicotinonitrile (C07), valproic acid (VA1), fluticasone (F), or derivatives or salts thereof.

In some embodiments, the disclosure relates to a process for engineering bone tissue comprising combining a sclerostin inhibitor disclosed herein, such as the compounds 6-((2-(pyrimidin-2-ylamino)ethyl)amino)nicotinonitrile (C07), valproic acid (VA1), fluticasone (F), or derivatives, and optionally a bone morphogenetic protein with a cell selected from the group consisting of osteogenic cells, pluripotent stem cells, mesenchymal cells, and embryonic stem cells.

Typically a sclerostin inhibitor disclosed herein is used locally such as injection percutaneously at any bone formation site (fracture, spine fusion delayed a day or several days after surgery) etc. The compound may also be bound to a matrix or scaffold and delivered with growth factors, cells (MSCs or others), or on a dry carrier matrix to direct local bone formation in the shape of the carrier/scaffold. Within certain embodiments, it is also contemplated that the sclerostin inhibitor is used in combination with other inhibitors that regulate BMP interactions, expression, or degradation such as a Smurf inhibitor and/or a JAB1 inhibitor.

### BRIEF DESCRIPTION OF THE SEVERAL VIEWS OF THE DRAWINGS

FIG. 1 illustrates the canonical Wnt/β-catenin pathway as a regulator of osteoblastogenesis and cross-talk with canonical BMP signaling. In canonical Wnt signaling, Wnt molecules bind to the Frizzled receptors and to the LRP5/6 co-receptors, thereby controlling the stability of cytoplasmic β-catenin. The importance of Wnt signaling in bone formation is illustrated by the low bone mass osteoporosis-pseudoglioma syndrome or high bone mass phenotype caused either by missense loss or gain of function mutations in LRP5, respectively. In the absence of Wnt ligands, β-catenin forms a complex with APC (adenomatous polyposis coli), axin, GSK3, and CK1 (casein kinase I). This complex facilitates the phosphorylation and subsequent proteasomal degradation of β-catenin. In the presence of Wnt ligands, however, this complex dissociates. Beta-catenin accumulates and translocates into the nucleus where it forms complexes with T-cell factor/lymphoid enhancer factor (TCF/Lef1) transcription factors leading to expression of various target genes. In osteoblasts, these genes enhance the proliferation, expansion, and survival of these cells, resulting in increased bone formation. The regulation of the Wnt pathway is ensured physiologically by the Wnt receptor antagonist sclerostin, which binds to the LRP5/6 receptor and inactivates Wnt signaling. Therefore, sclerostin leads to the direct inhibition of osteoblastogenesis.

FIG. 2 illustrates compound C07 having the chemical name 6-((2-(pyrimidin-2-ylamino)ethyl)amino)nicotinonitrile.

FIG. 3 shows data for using a Wnt reporter assay, Wnt3a response in a mouse myoblast cell line (C2C12). VA1 is valproic acid. F is fluticasone.

FIG. 4 shows data indicating mineralization of compounds via von Kossa staining by murine MSC cells at 10 days. NT is no treatment, AA is ascorbic acid, BGP is beta-glycerophosphate, DMSO is dimethyl sulfoxide.

FIG. 5 shows data on bone-inducing activity of the compound C07 in vivo. Compound was delivered in 100 uL at indicated concentration for each collagen disc in DMSO solvent before subcutaneous implantation.

FIG. 6 illustrates preparation of compounds disclosed herein using procedures as described or appropriately modified with appropriate starting materials in Yin et al, J Org Chem, 2007, 72, 4554-4557; Stroup et al. Org Lett, 2007, 9, 2039-2042; Santos et al. Molecules, 2018, 23, 2673 4; and Peng et al, Org Lett, 2006, 8, 395-398.

FIG. 7A shows data indicating locally delivered sclerostin inhibitor C07 produce successful spine fusions in vivo. Coronal μCT reconstructions of rabbit spines 6 weeks following posterolateral spine arthrodesis are shown. In rabbits that received C07 along with autologous iliac crest bone graft (ICBG), the posterolateral spine fusion rate was significantly increased compared to controls with ICBG alone (85% vs. 66%, P<0.05).

FIG. 7B shows data for rabbits that received VA1 with autologous ICBG, the fusion rate was also significantly increased compared to controls with ICBG alone (80% vs. 66%, P<0.05).

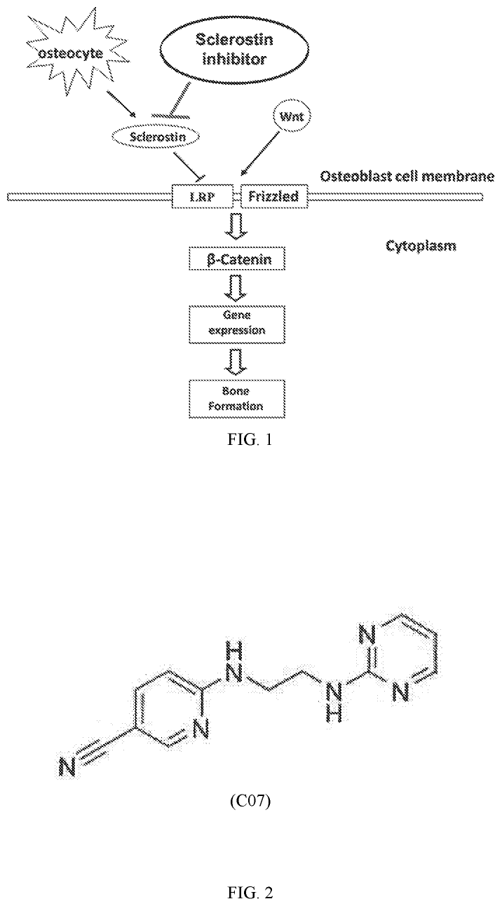

### DETAILED DISCUSSION

Before the present disclosure is described in greater detail, it is to be understood that this disclosure is not limited to particular embodiments described, and as such may, of course, vary. It is also to be understood that the terminology used herein is for the purpose of describing particular embodiments only, and is not intended to be limiting, since the scope of the present disclosure will be limited only by the appended claims.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described.

All publications and patents cited in this specification are herein incorporated by reference as if each individual publication or patent were specifically and individually indicated to be incorporated by reference and are incorporated herein by reference to disclose and describe the methods and/or materials in connection with which the publications are cited. The citation of any publication is for its disclosure prior to the filing date and should not be construed as an admission that the present disclosure is not entitled to antedate such publication by virtue of prior disclosure. Further, the dates of publication provided could be different from the actual publication dates that may need to be independently confirmed.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of medicine, organic chemistry, biochemistry, molecular biology, pharmacology, and the like, which are within the skill of the art. Such techniques are explained fully in the literature.

To the extent that chemical formula reported herein contain one or more chiral centers, the formula are intended to encompass all stable stereoisomers, enantiomers, and diastereomers. It is also understood that formula encompass all tautomeric forms.

It must be noted that, as used in the specification and the appended claims, the singular forms “a,” “an,” and “the” include plural referents unless the context clearly dictates otherwise.

“Ossification” refers to the process of laying down new bone by cells called osteoblasts. The term includes the growth in healing bone fractures treated by cast or by open reduction and stabilization by metal plate and screws. Ossification may also result in the formation of bone tissue in an extraskeletal location.

The term “bone morphogenetic protein” or “BMP” refers to any one of the family of growth factors or fragments thereof with the ability to induce the formation of bone and/or cartilage. The BMP receptors are typically serine-threonine kinases. It is not intended that BMP refer to any particular protein sequence and may or may not have certain glycosylation patterns attached thereto provided that the molecule has sufficient structural homology to any one of the known BMPs described below and retains some functional ability to promote bone growth, cartilage growth, or osteoblast differentiation. BMPs may be isolated from natural or non-natural sources, such as, but not limited to, recombinant or synthetic methods. References to BMPs generally or a specific BMP, e.g., BMP-2, includes recombinant or synthetically isolated versions unless otherwise provide for herein. Combinations of BMPs are contemplated. BMP-2 is known to induce bone and cartilage formation and play a role in osteoblast differentiation. BMP-3 is known to induce bone formation. BMP-4 is known to regulate the formation of teeth, limbs and bone from mesoderm and play a role in fracture repair. BMP-5 is known to function in cartilage development. BMP-6 is known to play a role in joint integrity and bone formation/repair. BMP-7 and BMP-9 are known to play a role in osteoblast differentiation. BMP-1 is a known metalloprotease that acts on procollagen I, II, and III and is involved in cartilage development.

As used herein, the term “derivative” refers to a structurally similar compound that retains sufficient functional attributes of the identified analogue. The derivative may be structurally similar because it is lacking one or more atoms, substituted, a salt, in different hydration/oxidation states, or because one or more atoms within the molecule are switched, such as, but not limited to, replacing a oxygen atom with a sulfur atom or replacing a amino group with a hydroxyl group. The derivative may be a prodrug. Derivatives may be prepare by any variety of synthetic methods or appropriate adaptations presented in synthetic or organic chemistry text books, such as those provide in March's Advanced Organic Chemistry: Reactions, Mechanisms, and Structure, Wiley, 6th Edition (2007) Michael B. Smith or Domino Reactions in Organic Synthesis, Wiley (2006) Lutz F. Tietze hereby incorporated by reference.

The term “substituted” refers to a molecule wherein at least one hydrogen atom is replaced with a substituent. When substituted, one or more of the groups are “substituents.” The molecule may be multiply substituted. In the case of an oxo substituent (“═O”), two hydrogen atoms are replaced. Example substituents within this context may include halogen, hydroxy, alkyl, alkoxy, nitro, cyano, oxo, carbocyclyl, carbocycloalkyl, heterocarbocyclyl, heterocarbocycloalkyl, aryl, arylalkyl, heteroaryl, heteroarylalkyl, —NRₐR_{b}, —NRₐC(═O)R_{b}, —NRₐC(═O)NRₐNR_{b}, —NRₐC(═O)OR_{b}, —NRₐSO₂R_{b}, —C(═O)Rₐ, —C(═O)ORₐ, —C(═O)NRₐR_{b}, —OC(═O)NRₐR_{b}, —ORₐ, —SRₐ, —SORₐ, —S(═O)₂Rₐ, —OS(═O)₂Rₐ and —S(═O)₂ORₐ. Rₐ and R_{b} in this context may be the same or different and independently hydrogen, halogen hydroxyl, alkyl, alkoxy, alkyl, amino, alkylamino, dialkylamino, carbocyclyl, carbocycloalkyl, heterocarbocyclyl, heterocarbocycloalkyl, aryl, arylalkyl, heteroaryl, and heteroarylalkyl.

As used herein, “subject” refers to any animal, preferably a human patient, livestock, or domestic pet.

As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression.

As used herein, the term “calcium phosphate(s)” refers to minerals containing calcium ions together with orthophosphates, metaphosphates or pyrophosphates and optionally hydroxide ions. Tricalcium phosphate is a calcium phosphate with formula Ca₃(PO₄)₂. The common mineral apatite has the basic formula Ca₅(PO₄)₃X, where X is an ion, typically a halogen or hydroxide ion, or a mixture. Hydroxyapatite refers to apatite where X is mainly hydroxide ion.

As used herein, “alkyl” means a noncyclic straight chain or branched, unsaturated or saturated hydrocarbon such as those containing from 1 to 10 carbon atoms. Representative saturated straight chain alkyls include methyl, ethyl, n-propyl, n-butyl, n-pentyl, n-hexyl, n-septyl, n-octyl, n-nonyl, and the like; while saturated branched alkyls include isopropyl, sec-butyl, isobutyl, tert-butyl, isopentyl, and the like. Unsaturated alkyls contain at least one double or triple bond between adjacent carbon atoms (referred to as an “alkenyl” or “alkynyl”, respectively). Representative straight chain and branched alkenyls include ethylenyl, propylenyl, 1-butenyl, 2-butenyl, isobutylenyl, 1-pentenyl, 2-pentenyl, 3-methyl-1-butenyl, 2-methyl-2-butenyl, 2,3-dimethyl-2-butenyl, and the like; while representative straight chain and branched alkynyls include acetylenyl, propynyl, 1-butynyl, 2-butynyl, 1-pentynyl, 2-pentynyl, 3-methyl-1-butynyl, and the like.

Non-aromatic mono or polycyclic alkyls are referred to herein as “carbocycles” or “carbocyclyl” groups. Representative saturated carbocycles include cyclopropyl, cyclobutyl, cyclopentyl, cyclohexyl, and the like; while unsaturated carbocycles include cyclopentenyl and cyclohexenyl, and the like.

“Heterocarbocycles” or heterocarbocyclyl” groups are carbocycles which contain from 1 to 4 heteroatoms independently selected from nitrogen, oxygen and sulfur which may be saturated or unsaturated (but not aromatic), monocyclic or polycyclic, and wherein the nitrogen and sulfur heteroatoms may be optionally oxidized, and the nitrogen heteroatom may be optionally quaternized. Heterocarbocycles include morpholinyl, pyrrolidinonyl, pyrrolidinyl, piperidinyl, hydantoinyl, valerolactamyl, oxiranyl, oxetanyl, tetrahydrofuranyl, tetrahydropyranyl, tetrahydropyridinyl, tetrahydroprimidinyl, tetrahydrothiophenyl, tetrahydrothiopyranyl, tetrahydropyrimidinyl, tetrahydrothiophenyl, tetrahydrothiopyranyl, and the like.

“Aryl” means an aromatic carbocyclic monocyclic or polycyclic ring such as phenyl or naphthyl. Polycyclic ring systems may, but are not required to, contain one or more non-aromatic rings, as long as one of the rings is aromatic.

As used herein, “heteroaryl” or “heteroaromatic” refers an aromatic heterocarbocycle having 1 to 4 heteroatoms selected from nitrogen, oxygen and sulfur, and containing at least 1 carbon atom, including both mono- and polycyclic ring systems. Polycyclic ring systems may, but are not required to, contain one or more non-aromatic rings, as long as one of the rings is aromatic. Representative heteroaryls are furyl, benzofuranyl, thiophenyl, benzothiophenyl, pyrrolyl, indolyl, isoindolyl, azaindolyl, pyridyl, quinolinyl, isoquinolinyl, oxazolyl, isooxazolyl, benzoxazolyl, pyrazolyl, imidazolyl, benzimidazolyl, thiazolyl, benzothiazolyl, isothiazolyl, pyridazinyl, pyrimidinyl, pyrazinyl, triazinyl, cinnolinyl, phthalazinyl, and quinazolinyl. It is contemplated that the use of the term “heteroaryl” includes N-alkylated derivatives such as a 1-methylimidazol-5-yl substituent.

As used herein, “heterocycle” or “heterocyclyl” refers to mono- and polycyclic ring systems having 1 to 4 heteroatoms selected from nitrogen, oxygen and sulfur, and containing at least 1 carbon atom. The mono- and polycyclic ring systems may be aromatic, non-aromatic or mixtures of aromatic and non-aromatic rings. Heterocycle includes heterocarbocycles, heteroaryls, and the like.

“Alkylthio” refers to an alkyl group as defined above attached through a sulfur bridge. An example of an alkylthio is methylthio, (i.e., —S—CH₃).

“Alkoxy” refers to an alkyl group as defined above attached through an oxygen bridge. Examples of alkoxy include, but are not limited to, methoxy, ethoxy, n-propoxy, i-propoxy, n-butoxy, s-butoxy, t-butoxy, n-pentoxy, and s-pentoxy. Preferred alkoxy groups are methoxy, ethoxy, n-propoxy, i-propoxy, n-butoxy, s-butoxy, t-butoxy.

“Alkylamino” refers an alkyl group as defined above attached through an amino bridge. An example of an alkylamino is methylamino, (i.e., —NH—CH₃).

“Alkanoyl” refers to an alkyl as defined above attached through a carbonyl bridge (i.e., —(C═O)alkyl).

“Alkylsulfonyl” refers to an alkyl as defined above attached through a sulfonyl bridge (i.e., —S(═O)₂alkyl) such as mesyl and the like, and “Arylsulfonyl” refers to an aryl attached through a sulfonyl bridge (i.e., —S(═O)₂aryl).

“Alkylsulfamoyl” refers to an alkyl as defined above attached through a sulfamoyl bridge (i.e., —S(═O)₂NHalkyl), and an “Arylsulfamoyl” refers to an alkyl attached through a sulfamoyl bridge (i.e., —S(═O)₂NHaryl).

“Alkylsulfinyl” refers to an alkyl as defined above with the indicated number of carbon atoms attached through a sulfinyl bridge (i.e. —S(═O)alkyl).

The terms “halogen” and “halo” refer to fluorine, chlorine, bromine, and iodine.

The term “bone graft composition” refers to materials that are substantially physiologically compatible when residing in bone area, void, or exterior site. In certain embodiments, the bone graft composition may be a bone graft matrix such as a collagen sponge or a mixture of polymers and salts.

When used in reference to compound(s) disclosed herein, “salts” refer to derivatives of the disclosed compound(s) where the parent compound is modified making acid or base salts thereof. Examples of salts include, but are not limited to, mineral or organic acid salts of basic residues such as amines, alkylamines, or dialkylamines; alkali or organic salts of acidic residues such as carboxylic acids; and the like.

### Small Molecule Inhibitors of Sclerostin to Improve Surgical Spine Fusions In Vivo

Spinal arthrodesis procedures are commonly performed for a wide range of pathology. Despite state-of-the-art surgical procedures, fusion failure rates still range from 10-40%. Failure to fuse is often associated with continued pain, worse outcomes, increased medication requirements, and possibly the cost of additional surgeries. Given the individual shortcomings of the currently available bone graft options, there is a clinical need for additional strategies that the surgeon can utilize in order to achieve consistently successful spinal fusions.

Promotion of canonical Wnt signaling through blocking the Wnt inhibitor sclerostin represents an opportunity to enhance bone formation locally. Ideally an anabolic bone forming effect can be achieved while simultaneously decreasing bone resorption. Sclerostin blocking strategies involve monoclonal antibodies (mAbs) delivered systemically are not ideal in local bone healing applications like posterolateral spine fusions. Systemic dosing, as compared to local delivery, typically requires higher dosing regimens and raises concern over the potential for off-target side effects. Thus, local delivery of small molecule inhibitors (SMIs) of sclerostin was examined. Ideally administration of a locally delivered anti-sclerostin SMI at the time of spinal arthrodesis surgery will result in a lower pseudarthrosis rate compared to autologous iliac crest bone grafting (the gold standard). A reduced spinal pseudarthrosis rate in subjects is preferred.

Small molecule inhibitors (SMIs) of sclerostin can enhance Wnt/beta-catenin and BMP signaling as well as promote mineralization in vitro. A locally delivered SMI of sclerostin promotes local bone formation within a spine fusion bed via enhancement of Wnt/beta-catenin in both migrating mesenchymal stem cells (MSCs) and resident osteoblasts at the fusion site while transiently inhibiting osteoclastic bone resorption. SMI are tested for their ability to induce ectopic de novo subcutaneous mineralization in a challenging rat model. Preferably, locally delivered anti-sclerostin SMIs enhances successful spine fusions.

### Sclerostin Inhibitors and Derivatives

Sclerostin inhibitors and derivatives may be used for bone growth and related applications. Derivatives of sclerostin inhibitors are further exemplified below.

In certain embodiments, derivatives of 6-((2-(pyrimidin-2-ylamino) ethyl) amino) nicotine nitrile (C07) are compounds of formula I,

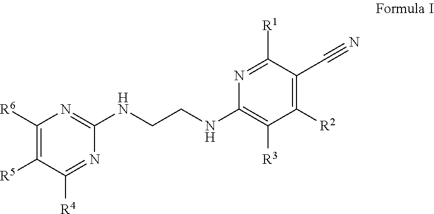

or salts thereof wherein,

R¹, R², R³, R⁴, R⁵, and R⁶ are, at each occurrence, the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R¹, R², R³, R⁴, R⁵, and R⁶ are optionally substituted with one or more, the same or different, R⁷;

R⁷ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R⁷ is optionally substituted with one or more, the same or different, R⁸; and

R⁸ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, derivatives of valproic acid are compounds of formula II,

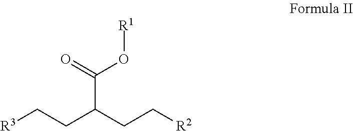

or salts thereof wherein,

R¹, R², and R³ are, at each occurrence, the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R¹, R², and R³ are optionally substituted with one or more, the same or different, R⁷;

R⁷ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R⁷ is optionally substituted with one or more, the same or different, R⁸; and

R⁸ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, derivatives of fluticasone are prodrugs or compounds of formula

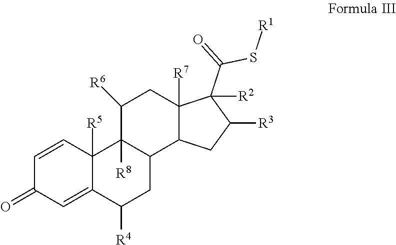

or salts thereof wherein,

R¹, R², R³, R⁴, R⁵, R⁶, R⁷, and R⁸ are each the same or different hydrogen, alkyl, alkenyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R¹, R², R³, R⁴, R⁵, R⁶, R⁷, and R⁸ are optionally substituted with one or more, the same or different, R¹⁰;

R¹⁰ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R¹⁰ is optionally substituted with one or more, the same or different, R¹¹; and

R¹¹ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

In certain embodiments, R¹ is an alkyl substituted with one or more halogens.

In certain embodiments, R² is hydroxy optionally substituted with an alkanoyl.

In certain embodiments, R⁴ and R⁸ are halogen.

In certain embodiments, R⁶ is hydroxy.

### Growth Factors

In some embodiments, the disclosure relates to the combined use of growth factor(s) and sclerostin inhibitors disclosed herein, such as the compounds 6-((2-(pyrimidin-2-ylamino)ethyl)amino)nicotinonitrile (C07), valproic acid (VA1), fluticasone (F), or derivatives and one or more growth factors in bone growth applications. Typically, the growth factor is a bone morphogenetic proteins (BMPs), including but not limited to, BMP-1, BMP-2, BMP-2A, BMP-2B, BMP-3, BMP-3b, BMP-4, BMP-5, BMP-6, BMP-7 (OP-1), BMP-8, BMP-8b, BMP-9, BMP-10, BMP-11, BMP-12, BMP-13, BMP-14, and BMP-15. BMPs act through specific transmembrane receptors located on cell surface of the target cells.

Non-limiting examples of additional suitable growth factors include osteogenin, insulin-like growth factor (IGF)-1, IGF-II, TGF-beta1, TGF-beta2, TGF-beta3, TGF-beta4, TGF-beta5, osteoinductive factor (OIF), basic fibroblast growth factor (bFGF), acidic fibroblast growth factor (aFGF), platelet-derived growth factor (PDGF), vascular endothelial growth factor (VEGF), growth hormone (GH), growth and differentiation factors (GDF)-5 through 9, and osteogenic protein-1 (OP-1). The growth factors may be isolated from synthetic methods, recombinant sources or may be purified from a biological sample. Preferably the growth factors are obtained from a recombinant technology and for clarity certain embodiments include rhBMP-2, rhBMP-4, rhBMP-6, rhBMP-7, and rhGDF-5, as disclosed, for example, in the U.S. Pat. Nos. 4,877,864; 5,013,649; 5,661,007; 5,688,678; 6,177,406; 6,432,919; 6,534,268, and 6,858,431; and in Wozney, J. M., et al. (1988) Science, 242(4885):1528-1534, all hereby incorporated by reference.

In a typical embodiment, a graft composition comprises a matrix, BMP-2, and a sclerostin inhibitor disclosed herein, such as the compounds 6-((2-(pyrimidin-2-ylamino)ethyl)amino)nicotinonitrile (C07), valproic acid (VA1), fluticasone (F), or derivatives or combinations of other growth factors such as GDF-5. In one embodiment, the matrix contains an effective amount of a BMP-2 protein, an rhBMP-2 protein, functional fragments thereof, or combinations thereof. For certain embodiments, the range of concentrations of BMP-2 may be about 1.0 to 4.0 mg/ml and GDF-5 concentrations may be 0.25 to 4.0 mg/ml. Although a graft matrix may be loaded during manufacturing, it is typically loaded just prior to implantation.

The transcription of human BMP-2 is 396 amino acids in length, localized to chromosome 20p12. BMP-2 belongs to the transforming growth factor-beta (TGF-beta) superfamily. The human amino acid sequence BMP-2 is SEQ ID NO: 1 shown below. Amino acids 38-268 are the TGF-beta propeptide domain, and 291-396 are the TGF-beta family N-terminal domain. Amino acids 283-396 are the mature peptide. The mature form of BMP-2 contains four potential N-linked glycosylation sites per polypeptide chain, and four potential disulfide bridges. (SEQ ID NO: 1) 1 MVAGTRCLLA LLLPQVLLGG AAGLVPELGR RKFAAASSGR PSSQPSDEVL SEFELRLLSM 61 FGLKQRPTPS RDAVVPPYML DLYRRHSGQP GSPAPDHRLE RAASRANTVR SFHHEESLEE 121 LPETSGKTTR RFFFNLSSIP TEEFITSAEL QVFREQMQDA LGNNSSFHHR INIYEIIKPA 181 TANSKFPVTR LLDTRLVNQN ASRWESFDVT PAVMRWTAQG HANHGFVVEV AHLEEKQGVS 241 KRHVRISRSL HQDEHSWSQI RPLLVTFGHD GKGHPLHKRE KRQAKHKQRK RLKSSCKRHP 301 LYVDFSDVGW NDWIVAPPGY HAFYCHGECP FPLADHLNST NHAIVQTLVN SVNSKIPKAC 361 CVPTELSAIS MLYLDENEKV VLKNYQDMVV EGCGCR.

In one embodiment, bone morphogenetic protein includes one of the following synthetic peptide fragments of BMP-2: (SEQ ID NO: 2) KIPKASSVPTELSAISTLYLDDD), SEQ ID NO: 3 (CCCCDDDSKIPKASSVPTELSAISTLYL, (SEQ ID NO: 4) C₁₆H₃₁O—NH—CCCCGGGSKIPKASSVPTELSAISTLYL which may be synthesized by FMOC/tBu solid-phase peptide synthesis.

BMP-7 also belongs to the TGF-beta superfamily. It induces cartilage and bone formation. The amino acid sequence of BMP-7 is SEQ ID NO: 5. (SEQ ID NO: 5) 1 MHVRSLRAAA PHSFVALWAP LFLLRSALAD FSLDNEVHSS FIHRRLRSQE RREMQREILS 61 ILGLPHRPRP HLQGKHNSAP MFMLDLYNAM AVEEGGGPGG QGFSYPYKAV FSTQGPPLAS 121 LQDSHFLTDA DMVMSFVNLV EHDKEFFHPR YHHREFRFDL SKIPEGEAVT AAEFRIYKDY 181 IRERFDNETF RISVYQVLQE HLGRESDLFL LDSRTLWASE EGWLVFDITA TSNHWVVNPR 241 HNLGLQLSVE TLDGQSINPK LAGLIGRHGP QNKQPFMVAF FKATEVHFRS IRSTGSKQRS 301 QNRSKTPKNQ EALRMANVAE NSSSDQRQAC KKHELYVSFR DLGWQDWIIA PEGYAAYYCE 361 GECAFPLNSY MNATNHAIVQ TLVHFINPET VPKPCCAPTQ LNAISVLYFD DSSNVILKKY 421 RNNVVRACGC H. Amino acids 1-29 are a potential signal sequence; 30-431 are the prepropeptide, and 293-431 are the mature protein. The mature form of BMP-7 contains four potential N-linked glycosylation sites per polypeptide chain, and four potential disulfide bridges.

### Graft Compositions

In some embodiments, the disclosure relates to graft compositions comprising sclerostin inhibitors disclosed herein, such as the compounds 6-((2-(pyrimidin-2-ylamino)ethyl)amino)nicotinonitrile (C07), valproic acid (VA1), fluticasone (F), or derivatives or salts thereof and optionally growth factor(s). In certain embodiments, these compositions may be created from polymers, demineralized bone matrix (DBM), bone granules, and ceramics such as calcium phosphates (e.g. hydroxyapatite and tricalcium phosphate), bioglass, and calcium sulphate. In certain embodiments, it is contemplated that the bone granules as autogenous, i.e., derived from the subject that is to receive the implanted bone graft. In certain embodiments, bone granules or demineralized (decalcified) bone matrix (DBM) are allogeneic, i.e., derived from somewhere other than the subject such as from another human or other animal. The grafts may contain carrier-beds of collagen or biodegradable polymers, antibacterials, bone morphogenetic proteins, and growth factors (platelet-derived growth factor, insulin-like growth factor, vascular endothelial and fibroblast growth factors), and bone marrow aspirate.

Demineralized bone matrix (DBM) typically contains collagen (mostly type I with some types IV and X), non-collagenous proteins and growth factors, a variable percent of residual calcium phosphate mineral. DBM is typically derived from bone morsellized to defined particles or fibers and subjected to acid demineralization followed by one or more rounds of freeze-drying, e.g., the mineral phase is extracted from the particulate whole donor bone with hydrochloric acid, leaving the organic matrix intact. The demineralized bone powder can be formulated into putties, pastes, flexible, or pre-formed strips by integration with a carrier, e.g., polymer, collagen, albumin, carboxymethyl cellulose, lecithin, hydrogel, gelatin, cancellous chips, alginate salt.

In certain embodiments, the disclosure relates to graft compositions comprising sclerostin inhibitor disclosed herein, such as the compounds 6-((2-(pyrimidin-2-ylamino)ethyl)amino)nicotinonitrile (C07), valproic acid (VA1), fluticasone (F), or derivatives or salts thereof covalently linked to bone graft compositions or scaffolds. In some embodiments, these compositions may be combined with growth factor(s).

Bioglass refers to materials of SiO₂, Na₂O, CaO and P₂O₅ in specific proportions. The proportions differ from the traditional soda-lime glasses in lower amounts of silica (typically less than 60 mol %), higher amounts of sodium and calcium, and higher calcium/phosphorus ratio. A high ratio of calcium to phosphorus promotes formation of apatite crystals; calcium and silica ions can act as crystallization nuclei. Some formulations bind to soft tissues and bone, some only to bone, some do not form a bond at all and after implantation get encapsulated with non-adhering fibrous tissue, and others are completely absorbed overtime. Mixtures of 35-60 mol % SiO₂, 10-50 mol % CaO, and 5-40 mol % Na₂O bond to bone and some formulations bond to soft tissues. Mixtures of >50 mol % SiO₂, <10 mol % CaO, <35 mol % Na₂O typically integrate within a month. Some CaO may be replaced with MgO and some Na₂O may be replaced with K₂O. Some CaO may be replaced with CaF₂.

In some embodiments, the disclosure relates to a graft composition comprising sclerostin inhibitor disclosed herein, such as the compounds 6-((2-(pyrimidin-2-ylamino)ethyl)amino)nicotinonitrile (C07), valproic acid (VA1), fluticasone (F), or derivatives and/or polysaccharides such as hyaluronate, cellulose or cellulose derivatives such as, but not limited to, hydroxypropyl cellulose, methyl cellulose, ethyl cellulose, and carboxymethyl cellulose. Typically, cellulose derivatives are used in graft compositions that produce a paste or putty.

In some embodiments, the disclosure relates to bone graft compositions comprising a bone morphogenetic protein and sclerostin inhibitor disclosed herein, such as the compounds 6-((2-(pyrimidin-2-ylamino)ethyl)amino)nicotinonitrile (C07), valproic acid (VA1), fluticasone (F), or derivatives and a graft matrix. The matrix is typically a polymer designed to hold bone compatible salts, such as calcium phosphates, for replacement during bone growth. An example is a bovine Type I collagen embedded with biphasic calcium phosphate granules. Optionally, matrix compositions may also include one or more agents that support the formation, development and growth of new bone, and/or the remodeling thereof. Typical examples of compounds that function in, such a supportive manner include extracellular matrix-associated bone proteins such as alkaline phosphatase, osteocalcin, bone sialoprotein (BSP) and osteocalcin, phosphoprotein (SPP)-1, type I collagen, fibronectin, osteonectin, thrombospondin, matrix-gla-protein, SPARC, and osteopontin.

In certain embodiments, the graft matrix can be made up of a hydrogel polymer. Typically, a hydrogel is made-up of acrylate polymers and copolymers substituted with an abundance of hydrophilic groups, such as terminal hydroxyl or carboxyl groups. In certain embodiments, the graft composition is biodegradable. In certain embodiments, the matrix comprises homopolymers and copolymers consisting of glycolide and lactide. For certain embodiments, the graft composition comprises a matrix of hydroxyethylmethacrylate or hydroxymethylmethyacrylate polymers containing hydroxyapatite in a mineral content approximately that of human bone. Such a composition may also be made with crosslinkers comprising an ester, anhydride, orthoester, amide, or peptide bond. In some embodiments, crosslinkers contain the following polymers: polyethylene glycol (PEG), polylactic acid, polyglycolide or combinations thereof.

In certain embodiments, graft comprises recombinant human platelet-derived growth factor (becaplermin).

In certain embodiments, graft is an antimicrobial silver wound dressing, silver-coated synthetic mesh, e.g., a synthetic layer of nylon, coated with silver.

In certain embodiments, graft comprises platelet rich plasma (PRP), derived from the blood of a subject after high-speed centrifugation or autologous conditioned plasma (ACP), removal of white blood cells. The blood or platelet rich plasma portion may be activated with various reagents to convert the blood protein fibrinogen into fibrin. This fibrin-rich gel-like substance is then immediately applied to the graft.

In certain embodiments, graft comprises bone marrow aspirate, e.g. derived via needle aspiration of bone marrow.

In certain embodiments, the bone graft comprises mesenchymal stem cells.

In certain embodiments, the bone graft comprises silicate and calcium phosphate combined with autologous bone marrow aspirate (BMA).

In certain embodiments, graft comprises blood mixed with microfibrillar collagen and thrombin.

In certain embodiments, the bone graft comprises beta tricalcium phosphate (β-TCP) combined with recombinant human platelet-derived growth factor BB (rhPDGF-BB).

In certain embodiments, the bone graft comprises Type I bovine collagen and hydroxyapatite mixed with bone marrow aspirate.

In certain embodiments, the graft composition may contain one or more antibiotics and/or anti-inflammatory agents. Suitable antibiotics include, without limitation, nitroimidazole antibiotics, tetracyclines, penicillins, cephalosporins, carbopenems, aminoglycosides, macrolide antibiotics, lincosamide antibiotics, 4-quinolones, rifamycins and nitrofurantoin. Suitable specific compounds include, without limitation, ampicillin, amoxicillin, benzylpenicillin, phenoxymethylpenicillin, bacampicillin, pivampicillin, carbenicillin, cloxacillin, cyclacillin, dicloxacillin, methicillin, oxacillin, piperacillin, ticarcillin, flucloxacillin, cefuroxime, cefetamet, cefetrame, cefixine, cefoxitin, ceftazidime, ceftizoxime, latamoxef, cefoperazone, ceftriaxone, cefsulodin, cefotaxime, cephalexin, cefaclor, cefadroxil, cefalothin, cefazolin, cefpodoxime, ceftibuten, aztreonam, tigemonam, erythromycin, dirithromycin, roxithromycin, azithromycin, clarithromycin, clindamycin, paldimycin, lincomycirl, vancomycin, spectinomycin, tobramycin, paromomycin, metronidazole, tinidazole, ornidazole, amifloxacin, cinoxacin, ciprofloxacin, difloxacin, enoxacin, fleroxacin, norfloxacin, ofloxacin, temafloxacin, doxycycline, minocycline, tetracycline, chlortetracycline, oxytetracycline, methacycline, rolitetracyclin, nitrofurantoin, nalidixic acid, gentamicin, rifampicin, amikacin, netilmicin, imipenem, cilastatin, chloramphenicol, furazolidone, nifuroxazide, sulfadiazin, sulfametoxazol, bismuth subsalicylate, colloidal bismuth subcitrate, gramicidin, mecillinam, cloxiquine, chlorhexidine, dichlorobenzylalcohol, methyl-2-pentylphenol or any combination thereof.

Suitable anti-inflammatory compounds include both steroidal and non-steroidal structures. Suitable non-limiting examples of steroidal anti-inflammatory compounds are corticosteroids such as hydrocortisone, cortisol, hydroxyltriamcinolone, alpha-methyl dexamethasone, dexamethasone-phosphate, beclomethasone dipropionates, clobetasol valerate, desonide, desoxymethasone, desoxycorticosterone acetate, dexamethasone, dichlorisone, diflorasone diacetate, diflucortolone valerate, fluadrenolone, fluclorolone acetonide, fludrocortisone, flumethasone pivalate, fluosinolone acetonide, fluocinonide, flucortine butylesters, fluocortolone, fluprednidene (fluprednylidene) acetate, flurandrenolone, halcinonide, hydrocortisone acetate, hydrocortisone butyrate, methylprednisolone, triamcinolone acetonide, cortisone, cortodoxone, flucetonide, fludrocortisone, difluorosone diacetate, fluradrenolone, fludrocortisone, diflurosone diacetate, fluocinolone, fluradrenolone acetonide, medrysone, amcinafel, amcinafide, betamethasone and the balance of its esters, chloroprednisone, chlorprednisone acetate, clocortelone, clescinolone, dichlorisone, diflurprednate, flucloronide, flunisolide, fluoromethalone, fluperolone, fluprednisolone, hydrocortisone valerate, hydrocortisone cyclopentylpropionate, hydrocortamate, meprednisone, paramethasone, prednisolone, prednisone, beclomethasone dipropionate, and triamcinolone. Mixtures of the above steroidal anti-inflammatory compounds may also be used.

Non-limiting examples of non-steroidal anti-inflammatory compounds include nabumetone, celecoxib, etodolac, nimesulide, apasone, gold, oxicams, such as piroxicam, isoxicam, meloxicam, tenoxicam, sudoxicam, the salicylates, such as aspirin, disalcid, benorylate, trilisate, safapryn, solprin, diflunisal, and fendosal; the acetic acid derivatives, such as diclofenac, fenclofenac, indomethacin, sulindac, tolmetin, isoxepac, furofenac, tiopinac, zidometacin, acematacin, fentiazac, zomepirac, clindanac, oxepinac, felbinac, and ketorolac; the fenamates, such as mefenamic, meclofenamic, flufenamic, niflumic, and tolfenamic acids; the propionic acid derivatives, such as ibuprofen, naproxen, benoxaprofen, flurbiprofen, ketoprofen, fenoprofen, fenbufen, indopropfen, pirprofen, carprofen, oxaprozin, pranoprofen, miroprofen, tioxaprofen, suprofen, alminoprofen, and tiaprofenic; and the pyrazoles, such as phenylbutazone, oxyphenbutazone, feprazone, azapropazone, and trimethazone.

### Bone Grafting Methods

Bone grafting is possible because bone tissue, unlike most other tissues, has the ability to regenerate if provided the space into which to grow with appropriate chemical signals. With regard to synthetic grafts, as native bone grows, it typically replaces most or all of the artificial graft material, resulting in an integrated region of new bone. However, with regard to certain embodiments of the disclosure, it is not intended that new bone must remove all artificial material. In addition, with regard to certain embodiments of the disclosure, it is not intended that graft location need contact any other bone of the skeletal system.

In certain embodiments, the disclosure relates to a method of forming bone comprising implanting a graft composition comprising a sclerostin inhibitor disclosed herein, such as the compounds 6-((2-(pyrimidin-2-ylamino)ethyl)amino)nicotinonitrile (C07), valproic acid (VA1), fluticasone (F), or derivatives or salts thereof, in a subject. In certain embodiments, the disclosure relates to methods of forming bone comprising implanting a graft composition comprising a bone morphogenetic protein and sclerostin inhibitors disclosed herein, such as the compounds 6-((2-(pyrimidin-2-ylamino)ethyl)amino)nicotinonitrile (C07), valproic acid (VA1), fluticasone (F), or derivatives, in a subject. The graft may be the result of a void created by surgical removal or created as a result of an attempt to correct a physical abnormality of a bone, such as but not limited to, cranial bones; frontal, parietal, temporal, occipital, sphenoid, ethmoid; facial bones; mandible, maxilla, palatine, zygomatic, nasal, lacrimal, vomer, inferior nasal conchae; shoulder girdle; scapula or shoulder blade, clavicle or collarbone; in the thorax; sternum, manubrium, gladiolus, and xiphoid process, ribs; in the vertebral column; cervical vertebrae, thoracic vertebrae; lumbar vertebrae; in the arms, humerus, radius, ulna; in the pelvis; coccyx; sacrum, hip bone (innominate bone or coxal bone); in the legs; femur, patella, tibia, and fibula. It is contemplated that the graft may be added for cosmetic purposes, e.g., cheek augmentation. In the case of a broken bone or removal of a bone during surgery, it may be desirable to secure movement of bone structure with a fixation system and remove the system after bone forms in the implanted graft.

With regard to prostheses, it may be desirable to grow bone between existing bone and an implanted device, or in preparation of an implanted device, such as in the case of a hip replacement, knee replacement, and dental implant, i.e., artificial tooth root used to support restorations that resemble a tooth or group of teeth.

In some embodiments, the disclosure relates to three-dimensional structures made of biocompatible and biodegradable bone graft materials in the shape of the bone infused with compositions disclosed herein to promote bone growth. Implants can be used to support a number of prostheses. A typical implant consists of a titanium device. In certain embodiments, the graft compositions disclosed herein contain implants.

With regard to a sinus augmentation or alveolar ridge augmentation, surgery may be performed as an outpatient under general anesthesia, oral conscious sedation, nitrous oxide sedation, intravenous sedation or under local anesthesia. Bone grafting is used in cases where there is a lack of adequate maxillary or mandibular bone in terms of depth or thickness. Sufficient bone is needed in three dimensions to securely integrate with the root-like implant. Improved bone height is important to assure ample anchorage of the root-like shape of the implant.

In a typical procedure, the clinician creates a large flap of the gingiva or gum to fully expose the bone at the graft site, performs one or several types of block and onlay grafts in and on existing bone, then installs a membrane designed to repel unwanted infection-causing bacteria. Then the mucosa is carefully sutured over the site. Together with a course of systemic antibiotics and topical antibacterial mouth rinses, the graft site is allowed to heal. The bone graft produces live vascular bone and is therefore suitable as a foundation for the dental implants.

In certain embodiments, the disclosure relates to methods of performing spinal fusion using compositions disclosed herein. Typically, this procedure is used to eliminate the pain caused by abnormal motion of the vertebrae by immobilizing the vertebrae themselves. Spinal fusion is often done in the lumbar region of the spine, but the term is not intended to be limited to method of fusing lumbar vertebrae. Patients desiring spinal fusion may have neurological deficits or severe pain, which has not responded to conservative treatment. Conditions where spinal fusion may be considered include, but are not limited to, degenerative disc disease, spinal disc herniation, discogenic pain, spinal tumor, vertebral fracture, scoliosis, kyphosis (i.e, Scheuermann's disease), spondylolisthesis, or spondylosis.

In certain embodiments, different methods of lumbar spinal fusion may be used in conjunction with each other. In one method, one places the bone graft between the transverse processes in the back of the spine. These vertebrae are fixed in place with screws and/or wire through the pedicles of each vertebra attaching to a metal rod on each side of the vertebrae. In another method, one places the bone graft between the vertebrae in the area usually occupied by the intervertebral disc. In preparation for the spinal fusion, the disc is removed entirely. A device may be placed between the vertebra to maintain spine alignment and disc height. The intervertebral device may be made from either plastic or titanium or other suitable material. The fusion then occurs between the endplates of the vertebrae. Using both types of fusion is contemplated.

### Cartilage Repair

Usami et al. report manipulation of Wnt signaling causes or ameliorates articular cartilage degeneration in rodent osteoarthritis models. Lab Invest, 2016,96(2):186-196. Cartilage is typically composed of chondroblasts, Type I and Type II collagen fibers, elastin fibers, and proteoglycans. Typical locations within the human body to find cartilage are the joints between bones, the ear, the nose, the elbow, the knee, the ankle, and the intervertebral discs. Cartilage can become damaged because of trauma or disease. In some embodiments, the disclosure relates to using sclerostin inhibitors disclosed herein, such as the compounds 6-((2-(pyrimidin-2-ylamino)ethyl)amino)nicotinonitrile (C07), valproic acid (VA1), fluticasone (F), or derivatives for the repair or regeneration of cartilage such as articular cartilage repair or regeneration or intervertebral disc cartilage repair or regeneration.

Articular cartilage repair is typically done to restore the cartilage on the surface of a bone, i.e., hyaline cartilage. Osteochondrial autografts or allografts may be performed. In certain embodiments, the disclosure contemplates methods of cartilage repair comprising transplanting sections of cartilage and/or bone to a location where cartilage and/or bone was removed and placing a sclerostin inhibitor disclosed herein, such as the compounds 6-((2-(pyrimidin-2-ylamino)ethyl)amino)nicotinonitrile (C07), valproic acid (VA1), fluticasone (F), or derivatives or salt thereof about the surrounding area, e.g., by injections at the site of transplantation. Bone with its cartilage covering may be removed from the same or a different joint and replanted into the hole left from removing degraded bone and cartilage. The transplanted bone and cartilage are typically taken from areas of low stress.

In autologous chondrocyte implantation, cartilage cells are typically extracted arthroscopically from normal articular cartilage of the subject that is located in a nonload-bearing area, e.g., the intercondylar notch or the superior ridge of the femoral condyles, and the cells are replicated, in vitro, in the presence of growth factors. In certain embodiments, the disclosure relates to replicating cartilage cells comprising mixing hyaline cartilage and a compound disclosed herein such as sclerostin inhibitors disclosed herein, such as the compounds 6-((2-(pyrimidin-2-ylamino)ethyl)amino)nicotinonitrile (C07), valproic acid (VA1), fluticasone (F), or derivatives or salt thereof, under conditions such that the cartilage cells replicate. Typically, this is done by adding other growth factors to the cartilage replicating medium, e.g., cartilage-derived morphogenetic proteins and/or BMP proteins. The replicated chondrocytes are implanted to the desired area, e.g., injected about the site of the area for repair optionally in combination with either a membrane or a matrix comprising growth factors such as a CDMP, BMP protein or a compound disclosed herein.

Repair of articular cartilage may be performed by marrow stimulating procedures sometimes referred to as microfracture surgery. Damaged cartilage is typically ablated by, e.g., drilling or pounding, exposing the underlying bone—sometimes referred to as a microfracture. The subchondal bone typically generates a blood clot followed by cartilage regeneration. In some embodiments the disclosure relates to methods of generating cartilage by disrupting bone underlying articular cartilage and placing a compound disclosed herein about the area of disruption, e.g., by injecting sclerostin inhibitors disclosed herein, such as the compounds 6-((2-(pyrimidin-2-ylamino)ethyl)amino)nicotinonitrile (C07), valproic acid (VA1), fluticasone (F), or derivatives or salt thereof about the site of disrupted bone for the improved repair or regeneration of cartilage optionally in combination with a growth factor such as a CDMP and/or BMP protein. Alternatively, it is contemplated that the compounds are administered to the subject in a pharmaceutical composition before, during or after the procedure. In another alternative, it is contemplated that a collagen matrix is implanted at the site of the exposed underlying bone to improve chondrogenic differentiation of mesenchymal stem cells. It is also contemplated that the subject may optionally be postoperative injected with compounds disclosed herein, hyaluronic acid, and/or mesenchymal stem cells, e.g., obtained from autologous peripheral blood progenitor cells.

Inflammation of the synovial membrane in a joint causes swelling and joint surface destruction. Removing excess fluid and material by a lavage or debridement frequently resolves arthritic knee inflammation and pain. In certain embodiments, the disclosure relates to the use of sclerostin inhibitor disclosed herein, such as the compounds 6-((2-(pyrimidin-2-ylamino)ethyl)amino)nicotinonitrile (C07), valproic acid (VA1), fluticasone (F), or derivatives or salt thereof before, during, or after a lavage or debridement inside a joint, e.g., arthroscopic lavage, arthroscopic debridement. In arthroscopic debridement, joint material or degenerative cartilage it typically removed by injecting a fluid and removing it with a vacuum.

An intervertebral disc (IVD) is found in between two vertebrae. The IVD contains different tissue types such as the annulus fibrosus (AF), the nucleus pulposus (NP), and end-plates. The AF is made up of mainly collagen type I. The amount of collagen type I decreases and collagen type II increase gradually nearer the NP which is mostly collagen type II dispersed within a proteoglycan-rich gelatinous matrix surrounding the NP.

Porous silk scaffolds may be used for a variety of tissue-engineering applications, such as the regeneration of bone and cartilage. Removal of sericin from silk reduces immunogenic responses. Silk may form a desired sponge-like structure by freeze-drying a silk solution. Bone marrow mesenchymal stem cells (BMSC) may be incorporated into porous silk scaffolds wrapped around a silicone NP substitute to form an artificial intervertebral disc. In certain embodiments, it is contemplated that sclerostin inhibitors disclosed herein, such as the compounds 6-((2-(pyrimidin-2-ylamino)ethyl)amino)nicotinonitrile (C07), valproic acid (VA1), fluticasone (F), or derivatives may be used to generate a matrix of annulus fibrosus by mixing with mesenchymal stem cells and growth factors. In certain embodiments, the disclosure contemplates implanting a fabricated intervertebral disc into a subject wherein the disc comprises annulus fibrosus tissue and placing a compound disclosed herein about the site of the implant location, e.g., by injection, optionally in combination with a growth factor such as a cartilage-derived morphogenetic protein (CDMP), e.g., CDMP-1 or CDMP-2, and/or bone morphogenetic proteins, e.g., BMP-7 or BMP-14. The fabricated disc may comprise a NP area with a hydrogel polymer/copolymer matrix or a collagen and/or hyaluronan and/or chondroitin-6-sulfate copolymer. A variety of stem cells, such as mesenchymal stem cells, synovium-derived stem cells (SDSCs), or notochord cells, may be used for rejuvenation of NP cells.

### Therapeutic Applications

In some embodiments, the disclosure relates to pharmaceutical compositions comprising sclerostin inhibitor disclosed herein, such as the compounds 6-((2-(pyrimidin-2-ylamino)ethyl)amino)nicotinonitrile (C07), valproic acid (VA1), fluticasone (F), or derivatives for therapeutic applications. In some embodiments, the disclosure relates to methods of treating bone degenerative disorders, such as osteoporosis, osteitis deformans (“Paget's disease of bone”), bone metastasis (with or without hypercalcaemia), multiple myeloma, primary hyperparathyroidism, or osteogenesis imperfecta. Osteoporosis is a disease of bones that leads to an increased risk of fracture. In osteoporosis, the bone mineral density (BMD) is reduced, bone microarchitecture is disrupted, and the amount and variety of proteins in bone is altered. Osteoporosis is most common in women after menopause, when it is called postmenopausal osteoporosis, but may also develop in men, and may occur in anyone in the presence of particular hormonal disorders and other chronic diseases or as a result of medications, specifically glucocorticoids, when the disease is called steroid- or glucocorticoid-induced osteoporosis (SIOP or GIOP).

In some embodiments, the disclosure relates to methods of treating bone degenerative disorders comprising administering an effective amount of a pharmaceutical composition comprising 6-((2-(pyrimidin-2-ylamino)ethyl)amino)nicotinonitrile, derivative, or salt thereof to a subject in need thereof.

In some embodiments, the disclosure relates to methods of treating bone degenerative disorders comprising administering an effective amount of a pharmaceutical composition comprising fluticasone, ester, derivative, or salt thereof to a subject in need thereof.

In some embodiments, the disclosure relates to methods of treating bone degenerative disorders comprising administering an effective amount of a pharmaceutical composition comprising fluticasone, derivative, or salt thereof to a subject in need thereof.

In some embodiments, the disclosure relates to methods of treating osteoporosis comprising administering an effective amount of a pharmaceutical composition comprising 6-((2-(pyrimidin-2-ylamino)ethyl)amino)nicotinonitrile, derivative, or salt thereof to a subject in need thereof.

In some embodiments, the disclosure relates to methods of treating osteoporosis comprising administering an effective amount of a pharmaceutical composition comprising fluticasone, ester, derivative, or salt thereof to a subject in need thereof.

In some embodiments, the disclosure relates to methods of treating osteoporosis comprising administering an effective amount of a pharmaceutical composition comprising fluticasone, derivative, or salt thereof to a subject in need thereof.

In some embodiments, the disclosure relates to methods of treating osteitis deformans comprising administering an effective amount of a pharmaceutical composition comprising 6-((2-(pyrimidin-2-ylamino)ethyl)amino)nicotinonitrile, derivative, or salt thereof to a subject in need thereof.

In some embodiments, the disclosure relates to methods of treating osteitis deformans comprising administering an effective amount of a pharmaceutical composition comprising fluticasone, ester, derivative, or salt thereof to a subject in need thereof.

In some embodiments, the disclosure relates to methods of treating osteitis deformans comprising administering an effective amount of a pharmaceutical composition comprising fluticasone, derivative, or salt thereof to a subject in need thereof.

In some embodiments, the disclosure relates to methods of treating bone metastasis comprising administering an effective amount of a pharmaceutical composition comprising 6-((2-(pyrimidin-2-ylamino)ethyl)amino)nicotinonitrile, derivative, or salt thereof to a subject in need thereof.

In some embodiments, the disclosure relates to methods of treating bone metastasis comprising administering an effective amount of a pharmaceutical composition comprising fluticasone, ester, derivative, or salt thereof to a subject in need thereof.

In some embodiments, the disclosure relates to methods of treating bone metastasis comprising administering an effective amount of a pharmaceutical composition comprising fluticasone, derivative, or salt thereof to a subject in need thereof.

In some embodiments, the disclosure relates to methods of treating multiple myeloma comprising administering an effective amount of a pharmaceutical composition comprising 6-((2-(pyrimidin-2-ylamino)ethyl)amino)nicotinonitrile, derivative, or salt thereof to a subject in need thereof.

In some embodiments, the disclosure relates to methods of treating multiple myeloma comprising administering an effective amount of a pharmaceutical composition comprising fluticasone, ester, derivative, or salt thereof to a subject in need thereof.

In some embodiments, the disclosure relates to methods of treating multiple myeloma comprising administering an effective amount of a pharmaceutical composition comprising fluticasone, derivative, or salt thereof to a subject in need thereof.

In some embodiments, the disclosure relates to methods of treating primary hyperparathyroidism comprising administering an effective amount of a pharmaceutical composition comprising 6-((2-(pyrimidin-2-ylamino)ethyl)amino)nicotinonitrile, derivative, or salt thereof to a subject in need thereof.

In some embodiments, the disclosure relates to methods of treating primary hyperparathyroidism comprising administering an effective amount of a pharmaceutical composition comprising fluticasone, ester, derivative, or salt thereof to a subject in need thereof.

In some embodiments, the disclosure relates to methods of treating primary hyperparathyroidism comprising administering an effective amount of a pharmaceutical composition comprising fluticasone, derivative, or salt thereof to a subject in need thereof.

In some embodiments, the disclosure relates to methods of treating osteogenesis imperfecta comprising administering an effective amount of a pharmaceutical composition comprising 6-((2-(pyrimidin-2-ylamino)ethyl)amino)nicotinonitrile, derivative, or salt thereof to a subject in need thereof.

In some embodiments, the disclosure relates to methods of treating osteogenesis imperfecta comprising administering an effective amount of a pharmaceutical composition comprising fluticasone, ester, derivative, or salt thereof to a subject in need thereof.

In some embodiments, the disclosure relates to methods of treating osteogenesis imperfecta m comprising administering an effective amount of a pharmaceutical composition comprising fluticasone, derivative, or salt thereof to a subject in need thereof.

Osteoporotic fractures are those that occur in situations where healthy people would not normally break a bone; they are therefore regarded as fragility fractures. Typical fragility fractures occur in the vertebral column, rib, hip and wrist. The diagnosis of osteoporosis can be made using conventional radiography by measuring the bone mineral density (BMD).

In some embodiments, the disclosure relates to treating bone degenerative disorders by administering pharmaceutical composition comprising sclerostin inhibitors disclosed herein, such as the compounds 6-((2-(pyrimidin-2-ylamino)ethyl)amino)nicotinonitrile (C07), valproic acid (VA1), fluticasone (F), or derivatives in combination with other agents, such as calcium carbonate and calcium citrate, vitamin D, cholecalciferol, 1,25-dihydroxy cholecalciferol, calcitriol, estrogen, testosterone, raloxifene, pamidronate, neridronate, olpadronate, alendronate, ibandronate, risedronate, zoledronate, etidronate, clodronate, or tiludronate.

### Formulations

Pharmaceutical compositions comprising sclerostin inhibitors disclosed herein, such as the compounds 6-((2-(pyrimidin-2-ylamino)ethyl)amino)nicotinonitrile (C07), valproic acid (VA1), fluticasone (F), or derivatives may be in the form of pharmaceutically acceptable salts, as generally described below. Some preferred, but non-limiting examples of suitable pharmaceutically acceptable organic and/or inorganic acids are hydrochloric acid, hydrobromic acid, sulfuric acid, nitric acid, acetic acid and citric acid, as well as other pharmaceutically acceptable acids known per se (for which reference is made to the references referred to below).

When the compounds of the disclosure contain an acidic group as well as a basic group, the compounds of the disclosure may also form internal salts, and such compounds are within the scope of the disclosure. When the compounds of the disclosure contain a hydrogen-donating heteroatom (e.g. NH), the disclosure also covers salts and/or isomers formed by transfer of said hydrogen atom to a basic group or atom within the molecule.

Pharmaceutically acceptable salts of the compounds include the acid addition and base salts thereof. Suitable acid addition salts are formed from acids which form non-toxic salts. Examples include the acetate, adipate, aspartate, benzoate, besylate, bicarbonate/carbonate, bisulphate/sulphate, borate, camsylate, citrate, cyclamate, edisylate, esylate, formate, fumarate, gluceptate, gluconate, glucuronate, hexafluorophosphate, hibenzate, hydrochloride/chloride, hydrobromide/bromide, hydroiodide/iodide, isethionate, lactate, malate, maleate, malonate, mesylate, methylsulphate, naphthylate, 2-napsylate, nicotinate, nitrate, orotate, oxalate, palmitate, pamoate, phosphate/hydrogen phosphate/dihydrogen phosphate, pyroglutamate, saccharate, stearate, succinate, tannate, tartrate, tosylate, trifluoroacetate and xinofoate salts. Suitable base salts are formed from bases which form non-toxic salts. Examples include the aluminium, arginine, benzathine, calcium, choline, diethylamine, diolamine, glycine, lysine, magnesium, meglumine, olamine, potassium, sodium, tromethamine and zinc salts. Hemisalts of acids and bases may also be formed, for example, hemisulphate and hemicalcium salts. For a review on suitable salts, see Handbook of Pharmaceutical Salts: Properties, Selection, and Use by Stahl and Wermuth (Wiley-VCH, 2002), incorporated herein by reference.

The compounds described herein may be administered in the form of prodrugs. A prodrug can include a covalently bonded carrier which releases the active parent drug when administered to a mammalian subject. Prodrugs can be prepared by modifying functional groups present in the compounds in such a way that the modifications are cleaved, either in routine manipulation or in vivo, to the parent compounds. Prodrugs include, for example, compounds wherein a hydroxyl group is bonded to any group that, when administered to a mammalian subject, cleaves to form a free hydroxyl group. Examples of prodrugs include, but are not limited to, acetate, formate and benzoate derivatives of alcohol functional groups in the compounds. Methods of structuring a compound as prodrugs can be found in the book of Testa and Mayer, Hydrolysis in Drug and Prodrug Metabolism, Wiley (2006). Typical prodrugs form the active metabolite by transformation of the prodrug by hydrolytic enzymes, the hydrolysis of amide, lactams, peptides, carboxylic acid esters, epoxides or the cleavage of esters of inorganic acids. It is well within the ordinary skill of the art to make an ester prodrug, e.g., acetyl ester of a free hydroxyl group. It is well known that ester prodrugs are readily degraded in the body to release the corresponding alcohol. See e.g., Imai, Drug Metab Pharmacokinet. (2006) 21(3):173-85, entitled “Human carboxylesterase isozymes: catalytic properties and rational drug design.”

Pharmaceutical compositions for use in the present disclosure typically comprise an effective amount of a compound and a suitable pharmaceutical acceptable carrier. The preparations may be prepared in a manner known per se, which usually involves mixing the at least one compound according to the disclosure with the one or more pharmaceutically acceptable carriers, and, if desired, in combination with other pharmaceutical active compounds, when necessary under aseptic conditions. Reference is made to U.S. Pat. Nos. 6,372,778, 6,369,086, 6,369,087 and 6,372,733 and the further references mentioned above, as well as to the standard handbooks, such as the latest edition of Remington's Pharmaceutical Sciences.

Generally, for pharmaceutical use, the compounds may be formulated as a pharmaceutical preparation comprising at least one compound and at least one pharmaceutically acceptable carrier, diluent or excipient and/or adjuvant, and optionally one or more further pharmaceutically active compounds.

The pharmaceutical preparations of the disclosure are preferably in a unit dosage form, and may be suitably packaged, for example in a box, blister, vial, bottle, sachet, ampoule or in any other suitable single-dose or multi-dose holder or container (which may be properly labeled); optionally with one or more leaflets containing product information and/or instructions for use. Generally, such unit dosages will contain between 1 and 1000 mg, and usually between 5 and 500 mg, of the at least one compound of the disclosure, e.g. about 10, 25, 50, 100, 200, 300 or 400 mg per unit dosage.

The compounds can be administered by a variety of routes including the oral, ocular, rectal, transdermal, subcutaneous, intravenous, intramuscular or intranasal routes, depending mainly on the specific preparation used. The compound will generally be administered in an “effective amount”, by which is meant any amount of a compound that, upon suitable administration, is sufficient to achieve the desired therapeutic or prophylactic effect in the subject to which it is administered. Usually, depending on the condition to be prevented or treated and the route of administration, such an effective amount will usually be between 0.01 to 1000 mg per kilogram body weight of the patient per day, more often between 0.1 and 500 mg, such as between 1 and 250 mg, for example about 5, 10, 20, 50, 100, 150, 200 or 250 mg, per kilogram body weight of the patient per day, which may be administered as a single daily dose, divided over one or more daily doses. The amount(s) to be administered, the route of administration and the further treatment regimen may be determined by the treating clinician, depending on factors such as the age, gender and general condition of the patient and the nature and severity of the disease/symptoms to be treated. Reference is made to U.S. Pat. Nos. 6,372,778, 6,369,086, 6,369,087 and 6,372,733 and the further references mentioned above, as well as to the standard handbooks, such as the latest edition of Remington's Pharmaceutical Sciences.

For an oral administration form, the compound can be mixed with suitable additives, such as excipients, stabilizers or inert diluents, and brought by means of the customary methods into the suitable administration forms, such as tablets, coated tablets, hard capsules, aqueous, alcoholic, or oily solutions. Examples of suitable inert carriers are gum arabic, magnesia, magnesium carbonate, potassium phosphate, lactose, glucose, or starch, in particular, corn starch. In this case, the preparation can be carried out both as dry and as moist granules. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms. As immediate release tablets, these compositions may contain microcrystalline cellulose, dicalcium phosphate, starch, magnesium stearate and lactose and/or other excipients, binders, extenders, disintegrants, diluents and lubricants known in the art.

When administered by nasal aerosol or inhalation, the compositions may be prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. Suitable pharmaceutical formulations for administration in the form of aerosols or sprays are, for example, solutions, suspensions or emulsions of the compounds of the disclosure or their physiologically tolerable salts in a pharmaceutically acceptable solvent, such as ethanol or water, or a mixture of such solvents. If required, the formulation may additionally contain other pharmaceutical auxiliaries such as surfactants, emulsifiers and stabilizers as well as a propellant.

For subcutaneous or intravenous administration, the compounds, if desired with the substances customary therefore such as solubilizers, emulsifiers or further auxiliaries are brought into solution, suspension, or emulsion. The compounds may also be lyophilized and the lyophilizates obtained used, for example, for the production of injection or infusion preparations. Suitable solvents are, for example, water, physiological saline solution or alcohols, e.g. ethanol, propanol, glycerol, sugar solutions such as glucose or mannitol solutions, or mixtures of the various solvents mentioned. The injectable solutions or suspensions may be formulated according to known art, using suitable non-toxic, parenterally-acceptable diluents or solvents, such as mannitol, 1,3-butanediol, water, Ringer's solution or isotonic sodium chloride solution, or suitable dispersing or wetting and suspending agents, such as sterile, bland, fixed oils, including synthetic mono- or diglycerides, and fatty acids, including oleic acid.

When rectally administered in the form of suppositories, the formulations may be prepared by mixing the compounds of formula I with a suitable non-irritating excipient, such as cocoa butter, synthetic glyceride esters or polyethylene glycols, which are solid at ordinary temperatures, but liquefy and/or dissolve in the rectal cavity to release the drug.

In certain embodiments, it is contemplated that these compositions can be extended release formulations. Typical extended release formations utilize an enteric coating. Typically, a barrier is applied to oral medication that controls the location in the digestive system where it is absorbed. Enteric coatings prevent release of medication before it reaches the small intestine. Enteric coatings may contain polymers of polysaccharides, such as maltodextrin, xanthan, scleroglucan dextran, starch, alginates, pullulan, hyaloronic acid, chitin, chitosan and the like; other natural polymers, such as proteins (albumin, gelatin etc.), poly-L-lysine; sodium poly(acrylic acid); poly(hydroxyalkylmethacrylates) (for example poly(hydroxyethylmethacrylate)); carboxypolymethylene (for example Carbopol™); carbomer; polyvinylpyrrolidone; gums, such as guar gum, gum arabic, gum karaya, gum ghatti, locust bean gum, tamarind gum, gellan gum, gum tragacanth, agar, pectin, gluten and the like; poly(vinyl alcohol); ethylene vinyl alcohol; polyethylene glycol (PEG); and cellulose ethers, such as hydroxymethylcellulose (HMC), hydroxyethylcellulose (HEC), hydroxypropylcellulose (HPC), methylcellulose (MC), ethylcellulose (EC), carboxyethylcellulose (CEC), ethylhydroxyethylcellulose (EHEC), carboxymethylhydroxyethylcellulose (CMHEC), hydroxypropylmethyl-cellulose (HPMC), hydroxypropylethylcellulose (HPEC) and sodium carboxymethylcellulose (Na CMC); as well as copolymers and/or (simple) mixtures of any of the above polymers. Certain of the above-mentioned polymers may further be crosslinked by way of standard techniques.

The choice of polymer will be determined by the nature of the active ingredient/drug that is employed in the composition of the disclosure as well as the desired rate of release. In particular, it will be appreciated by the skilled person, for example in the case of HPMC, that a higher molecular weight will, in general, provide a slower rate of release of drug from the composition. Furthermore, in the case of HPMC, different degrees of substitution of methoxyl groups and hydroxypropoxyl groups will give rise to changes in the rate of release of drug from the composition. In this respect, and as stated above, it may be desirable to provide compositions of the disclosure in the form of coatings in which the polymer carrier is provided by way of a blend of two or more polymers of, for example, different molecular weights in order to produce a particular required or desired release profile.

Microspheres of polylactide, polyglycolide, and their copolymers poly(lactide-co-glycolide) may be used to form sustained-release protein delivery systems. Proteins can be entrapped in the poly(lactide-co-glycolide) microsphere depot by a number of methods, including formation of a water-in-oil emulsion with water-borne protein and organic solvent-borne polymer (emulsion method), formation of a solid-in-oil suspension with solid protein dispersed in a solvent-based polymer solution (suspension method), or by dissolving the protein in a solvent-based polymer solution (dissolution method). One can attach poly(ethylene glycol) to proteins (PEGylation) to increase the in vivo half-life of circulating therapeutic proteins and decrease the chance of an immune response.

### Examples

In Vitro Screening of Small Molecules that Reverse Sclerostin-Mediated Inhibition of Wnt Signaling and Promote Osteogenesis

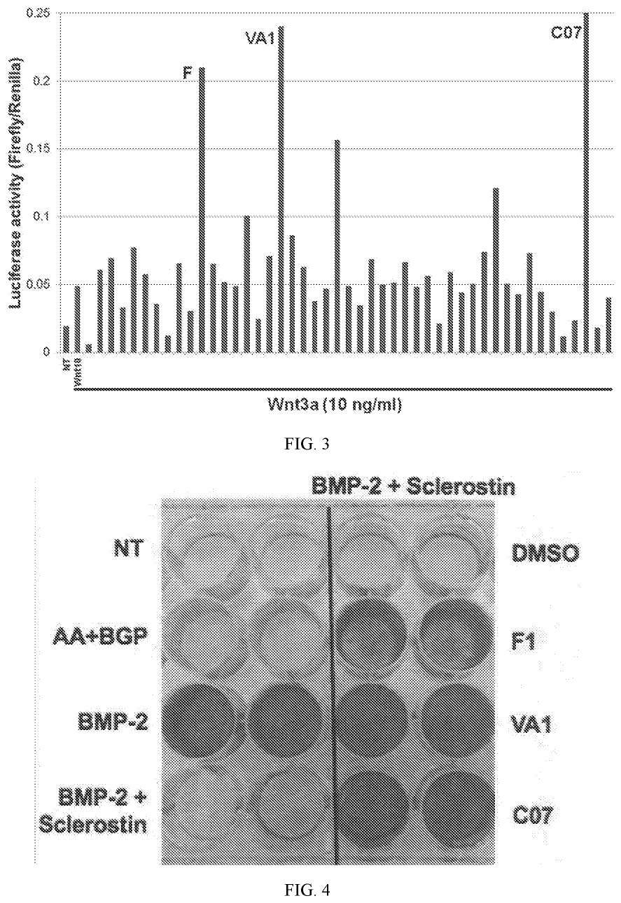

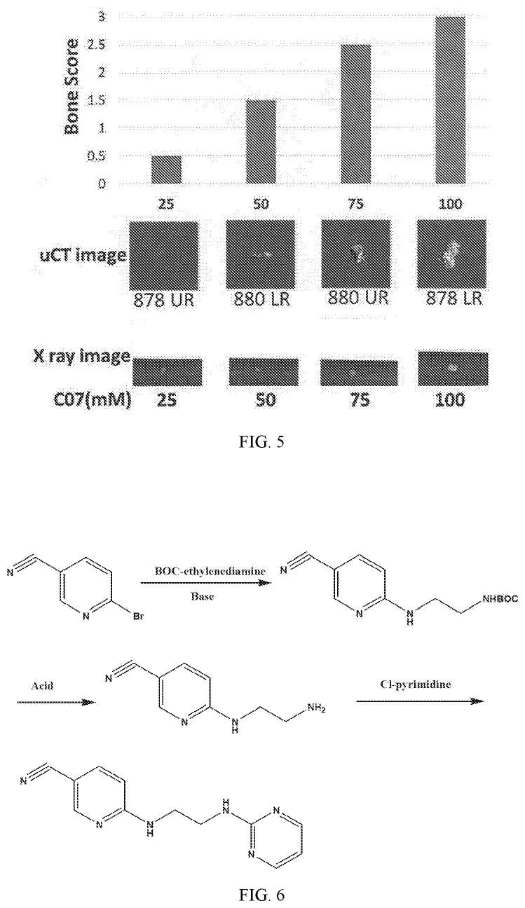

Receptor structure focused docking were performed using solution NMR PDB structure of sclerostin (ID 2K8P) to analyze the binding modes and their estimated affinities. Each compound was docked against both the target region of sclerostin. The predicted binding energy from the dockings provided a ranking of compounds based on their binding affinities. Select compounds were tested for their ability to enhance canonical Wnt signaling in vitro. The Wnt-specific TCF/LEF-driven Cignal™ reporter system (Qiagen) was optimized for Wnt3a response in a mouse myoblast cell line (C2C12), which were stimulated towards the osteoblastic phenotype (FIG. 3). The mouse-derived C2C12 myoblasts served as an experimentally tractable model system for investigating the molecular basis of transdifferentiation toward the osteoblastic phenotype. As a cell-based assay. LEF/TCF-driven-specific luciferase reporter plasmid was used to monitor transcriptional activity driven by activated Wnt pathway in C2C12 cells. To select a sub-optimal dose of Wnt3a for studying the potentiating effect of selected compounds, the reporter assay was performed with lower Wnt3a concentrations ranging from 0 to 80 ng/ml. A sub-optimal dose (10 ng/ml) of Wnt3a for activating the reporter assay was established. The results from this experiment allowed us to select a compound to assess the potentiating effects of compounds in subsequent experiments.

### Sclerostin Inhibitors Block Binding of Sclerostin to LRP5

To ensure that sclerostin was in fact being expressed in cell lines, we tested C2C12, MSC and MC3T3 cell lines. Western blots were performed using anti-sclerostin mAbs both with and without BMP-2 treatment. Sclerostin protein is present in these cells and BMP2 treatment resulted in increased protein levels. To confirm that identified compounds actually inhibits a sclerostin and LRP5/6 interaction, an in vitro binding assay was optimized with purified recombinant sclerostin and LRP5 proteins. Sclerostin and LRP5 was labeled with 125-Iodine and biotin, respectively. Constant amounts of biotin-labeled LRP5 (0.1 ug) and 125-Iodine-labeled sclerostin (20,000 cpm) were incubated with or without varying concentrations of unlabeled-sclerostin+/−various concentrations of compounds for 30 min at room temperature in a 100 uL assay buffer. Biotin-labeled LRP5 was pulled down with a 20 uL slurry of neutravidin-agarose beads. The counts associated with pellets were counted in a Beckman 4000 Gamma counter. Unlabeled sclerostin competed off about 90% of labeled-sclerostin. Data indicates that sclerostin and LRP5 interaction is saturable and concentration dependent in the binding assay. The compounds competed with Sclerostin to prevent up to 40% of the labeled Sclerostin from binding to LRP5, confirming that certain compounds significantly disrupt sclerostin binding to LRP5.

### Expression of BMP-2-Induced Genes in Wnt Pathway is Enhanced by Sclerostin Inhibitors

Experiments were performed to determine whether the compounds that enhanced Wnt-induced reporter activity would also exhibit potentiating activity on BMP-2-induced marker gene expression in Wnt pathway. the effectiveness compounds F, VA1 and C07 were determined at a concentration of 10 uM while keeping the BMP-2 concentration constant at 35 ng/ml by determining mRNA levels. In general, compounds caused elevation in the BMP-induced mRNA levels of AXIN2, Wnt1 and SOST compared to BMP-2 alone control when MC3T3 cells were treated for 48 hrs compared to sub-optimal BMP-2 (35 ng/ml) alone treatment. These genes in Wnt pathway were probably induced as a part of feed-back mechanism of BMP function.

### De Novo Ectopic Mineralization In Vivo

Sclerostin inhibitors were tested in a challenging in vivo subcutaneous ectopic mineralization model. Other than the osteoinductive BMPs, few if any proteins or small molecules are capable of inducing de novo ectopic mineralization in this rat model. Sclerostin inhibitors were loaded individually as standalone agents onto a plain collagen sponge (DSM) at 0, 10, 25, 50, 75, and 100 mM and then surgically implanted subcutaneously on the chest of 6-week-old male Sprague-Dawley rats for 4 weeks. A positive control of 10 μg of recombinant BMP-2 was also tested. Local subcutaneous delivery of both VA1 and C07 resulted in significant de novo ectopic mineralization as standalone agents, with C07 demonstrating a clearer dose-response. The lack of a clear dose-response for VA1 likely indicates that the carrier used in this study is not ideal and that the residence time in the local tissue is inconsistent.

### Sclerostin SMIs Enhance Spinal Fusion Rates In Vivo

C07 and VA1 were assessed for their ability to enhance spinal fusion rates in vivo using a validated rabbit model of posterolateral lumbar arthrodesis. Both were tested as standalone osteoinductive drugs, as well as in combination with autologous ICBG, using two separate doses (300 and 500 mM). All rabbits were euthanized six weeks following arthrodesis surgery and the spine fusion masses were assessed by both plain radiography and μCT. Successful fusion, defined as continuous bridging bone between the TPs, was assessed by two spine surgeons.

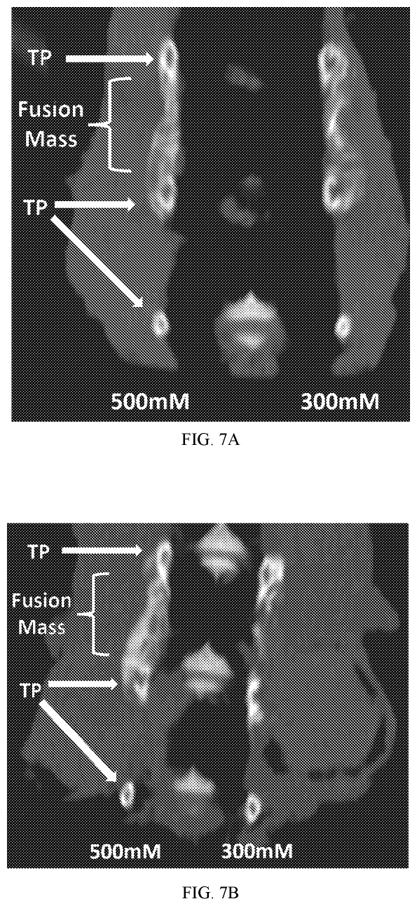

When C07 was used at a dose of 500 mM in combination with autologous iliac crest bone graft (ICBG), the posterolateral spine fusion rate was significantly increased compared to controls with ICBG alone (85% vs. 66%, P<0.05) (FIG. 7A). Similarly, when the higher dose of VA1 (500 mM) was used alongside autologous ICBG, the fusion rate was also significantly increased compared to controls with ICBG alone (80% vs. 66%, P<0.05) (FIG. 7B). When both C07 and VA1 were used at 500 mM as standalone drugs on a plain collagen sponge without ICBG, 33% and 17% of the spines successfully fused, respectively, which is significantly higher (P<0.05) than the 0% fusion rate in this model when the transverse processes are decorticated alone. Neither VA1 nor C07 at 300 mM showed any increase in spinal fusion rates compared to controls.

What is claimed is:

1. A graft composition comprising a sclerostin inhibitor or derivative or salt thereof.

2. The graft composition of claim 1 wherein the sclerostin inhibitor has Formula I: 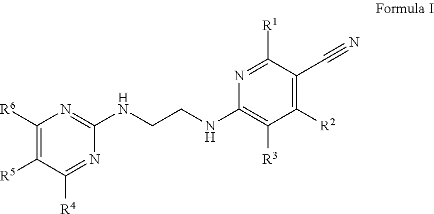
or salts thereof wherein,
R¹, R², R³, R⁴, R⁵, and R⁶ are, at each occurrence, the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R¹, R², R³, R⁴, R⁵, and R⁶ are optionally substituted with one or more, the same or different, R⁷;
R⁷ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R⁷ is optionally substituted with one or more, the same or different, R⁸; and
R⁸ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

3. The graft composition of claim 2 wherein the sclerostin inhibitor is 6-((2-(pyrimidin-2-ylamino)ethyl)amino)nicotinonitrile.

4. The graft composition of claim 1 wherein the sclerostin inhibitor has Formula II, 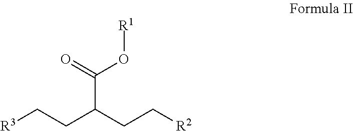
or salts thereof wherein,
R¹, R², and R³ are, at each occurrence, the same or different hydrogen, alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein each R¹, R², and R³ are optionally substituted with one or more, the same or different, R⁷;
R⁷ is alkyl, halogen, nitro, cyano, hydroxy, amino, mercapto, formyl, carboxy, alkanoyl, carbamoyl, alkoxy, alkylthio, alkylamino, (alkyl)₂amino, alkylsulfinyl, alkylsulfonyl, arylsulfonyl, carbocyclyl, aryl, or heterocyclyl, wherein R⁷ is optionally substituted with one or more, the same or different, R⁸; and
R⁸ is halogen, nitro, cyano, hydroxy, trifluoromethoxy, trifluoromethyl, amino, formyl, carboxy, carbamoyl, mercapto, sulfamoyl, methyl, ethyl, methoxy, ethoxy, acetyl, acetoxy, methylamino, ethylamino, dimethylamino, diethylamino, N-methyl-N-ethylamino, acetylamino, N-methylcarbamoyl, N-ethylcarbamoyl, N,N-dimethylcarbamoyl, N,N-diethylcarbamoyl, N-methyl-N-ethylcarbamoyl, methylthio, ethylthio, methylsulfinyl, ethylsulfinyl, mesyl, ethylsulfonyl, methoxycarbonyl, ethoxycarbonyl, N-methylsulfamoyl, N-ethylsulfamoyl, N,N-dimethylsulfamoyl, N,N-diethylsulfamoyl, N-methyl-N-ethylsulfamoyl, carbocyclyl, aryl, or heterocyclyl.

5. The graft of claim 4, wherein the compound is valproic acid or alkyl esters thereof.

6. The graft composition of claim 1 further comprising a collagen or hydrogel matrix.

7. A kit comprising a sclerostin inhibitor or derivative and a graft composition of claim 1.

8. The kit of claim 7 further comprising a growth factor or bone morphogenetic protein.

9. A method of forming bone or cartilage comprising implanting a bone graft composition comprising a sclerostin inhibitor or derivative optionally comprising a growth factor in a subject at a site of desired bone or cartilage growth.

10. The method of claim 9, wherein the growth factor is a bone morphogenetic protein selected from BMP-2, BMP-6, BMP-7, or BMP-9.

11. A method of forming bone comprising
a) implanting a bone graft composition optionally comprising a sclerostin inhibitor or derivative and optionally comprising a growth factor in a subject at a site of desired bone growth and
b) administering a pharmaceutical composition comprising a sclerostin inhibitor or derivative to the subject.

12-18. (canceled)
